(19) United States
(12) Patent Application Publication
(10) Pub. No.: US 2006/0063963 A1
(43) Pub. Date: Mar. 23, 2006

(54) Electromagnetic conditioning system for bathtub or shower enclosures

(75) Inventors: Henry Brunelle, Cap Rouge (CA); Stephane Baron, Charny (CA); Guy Baillargeon, St-Etienne de Lauzon (CA); Paul Demers, Breakeyville (CA); Guy Sirois, Breakeyville (CA)

Correspondence Address: OGILVY RENAULT LLP, 1981 MCGILL COLLEGE AVENUE, SUITE 1600, MONTREAL, QC, H3A2Y3, CA

(73) Assignee: Gestion Ultra Internationale Inc.

(21) Appl. No.: 11/229,549

(22) Filed: Sep. 20, 2005

(30) Foreign Application Priority Data
Sep. 22, 2004 (CA) 2,482,310

(51) Int. Cl. A61N 1/00

(52) U.S. Cl. 600/13

(57) ABSTRACT

An electromagnetic treatment system and method of use in combination with a bathtub or shower enclosure and preferably, but not exclusively, a therapeutic type enclosure provided with a therapeutic device to subject a user person to a therapeutic treatment, as well as an electromagnetic treatment. The electromagnetic treatment system comprises an electromagnetic coil attached to an outside surface of the wall of the therapeutic enclosure and surrounds a space in the enclosure where a user person is disposed to undergo an electromagnetic treatment. A pulsating voltage generator is connected to the electromagnetic coil for generating a pulsating electromagnetic about the coil. The electromagnetic field is oriented relative to the space. A control circuit controls the generator and a remote control, which is secured to the therapeutic enclosure and accessible to a user person, actuates the generator through the control circuit.

### FIELD OF THE INVENTION

The present invention relates to an electromagnetic treatment system in combination with a bathtub or shower enclosure and particularly, but not exclusively, a therapeutic type enclosure.

### BACKGROUND OF THE INVENTION

Electromagnetic treatment systems are known and wherein electromagnetic field generating devices are placed about a person's body to subject the body or a limb to a pulsed electromagnetic field, whereby to stimulate the human nervous system to a physiological exercise. Electromagnetic pulses induce physiological effects on biological cells found in the human body and improve the functioning thereof by the stimulation of blood circulation, relaxation of the nervous system and stimulation of normal cellular cell repair and regeneration. Such a system and devices are for example described in U.S. Pat. No. 5,620,463.

Some of the effects of electromagnetic treatment systems can be summarized by the following examples of benefits derived, but not limited thereto, and such as improved blood circulation and the expulsion of lactic acid through perspiration, the stimulation of cell generation as mentioned above, recalcification of bone cells, decalcification of soft tissues, the neutralization of free radicals, the reestablishment of electrical potential in cells, the stimulation of electrochemical cells, stress relief, favoring the absorption of vitamins and minerals and aligo-elements and the improvement of the absorption of molecules such as essential oils.

### SUMMARY OF INVENTION

It is a feature of the present invention to provide an electromagnetic treatment system in combination with a bathtub or shower enclosure and more specifically, but not exclusively, a therapeutic type enclosure, and wherein a user person can benefit from an electromagnetic treatment system as well as the other therapeutic benefits obtained from the bathtub or shower enclosure.

According to another feature of the present invention, there is provided a method of obtaining an electromagnetic treatment in a treatment area of one of a bathtub or shower enclosure, as well as the therapeutic benefits obtained from such enclosures.

According to the above features, the present invention provides an electromagnetic treatment system in combination with a bathing enclosure adapted to receive a user person. The electromagnetic treatment system comprises an electromagnetic coil attached to an outside surface of the wall of the bathing enclosure and surrounds a space in the enclosure where the user person is disposed to undergo an electromagnetic treatment. A pulsating voltage generator is connected to the electromagnetic coil for generating a pulsating electromagnetic field about the coil. The electromagnetic field is oriented relative to the space. Control circuit means is provided for controlling the generator. A remote control is secured to the enclosure and accessible to a user person for actuating the generator through the control circuit means.

According to a further broad aspect of the present invention, the method provides an electromagnetic treatment to a person disposed in a treatment area of one of a bathtub or shower enclosure. The method comprises the steps of providing such an enclosure with an electromagnetic coil attached to an outside surface of a wall thereof, and surrounding a space in the enclosure where a user person is disposed to undergo an electromagnetic treatment. A remote control is accessible to the user person. The method also comprises actuating a pulsating voltage generator by the remote control, whereby the coil generates a pulsating electromagnetic field into the space for a predetermined period of time to subject the user person to the electromagnetic treatment.

### BRIEF DESCRIPTION OF THE DRAWINGS

A preferred embodiment of the present invention will now be described with reference to the accompanying drawings, in which:

FIG. 1 is a perspective view showing a therapeutic bathtub provided with the electromagnetic treatment system of the present invention, as well as one or more therapeutic systems;

FIG. 2 is a block diagram showing some of the essential components of the electromagnetic treatment system;

FIG. 3 is a perspective view similar to FIG. 1, but showing the electromagnetic field pattern generated by the electromagnetic coil;

FIG. 4 is a perspective frontal view showing a shower enclosure equipped with an electromagnetic treatment area about which the electromagnetic treatment system is disposed for a user person to undergo such treatment; and

FIG. 5 is a rear fragmented perspective view of the shower enclosure in FIG. 4, showing the disposition of the electromagnetic coil.

### DESCRIPTION OF PREFERRED EMBODIMENTS

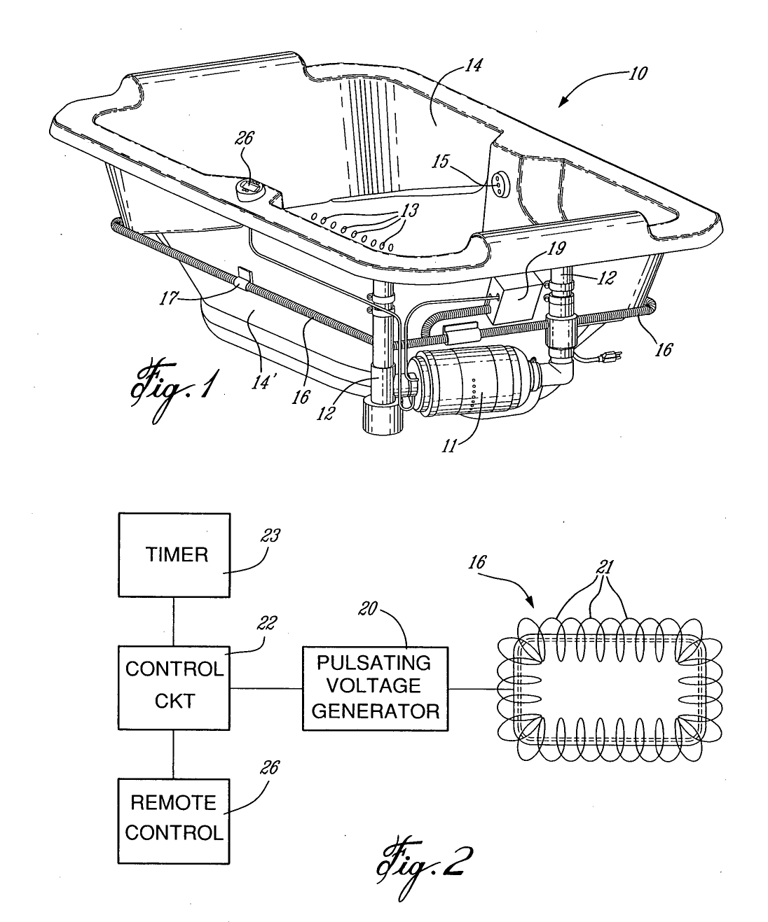

Referring now to the drawings, more particularly to FIGS. 1 to 3, there is shown generally at 10 a bathtub enclosure. The bathtub enclosure 10 may be a standard type bathtub in which water is admitted for a person to bathe therein. However, as illustrated in this figure, the bathtub enclosure is a therapeutic bathtub enclosure provided with a therapeutic treatment system, which is comprised of an air blower 11 connected to ducting 12, which is in communication with holes 13 providing jets within the side walls 14 of the bathtub enclosure, through which massaging air streams exit to provide a massage therapy to a user person disposed within the bathtub enclosure. Such therapeutic bathtub enclosures are well known in the art. The therapeutic bathtub is also provided with a chromo therapy treatment by the generation of light into the water through a LED lamp 15 located at a convenient location in the sidewall 14 of the bathtub enclosure.

The electromagnetic treatment system of the present invention is used in combination with such a therapeutic bathtub and comprises an electromagnetic coil 16, which is attached to the outer surface 14′ of the sidewalls 15 by means of attachment brackets 17. The electromagnetic coil 16 is an electrically conductive cable secured about the circumferential side wall of the bathtub and in a substantially horizontal plane and defines within this coil or loop a treatment area.

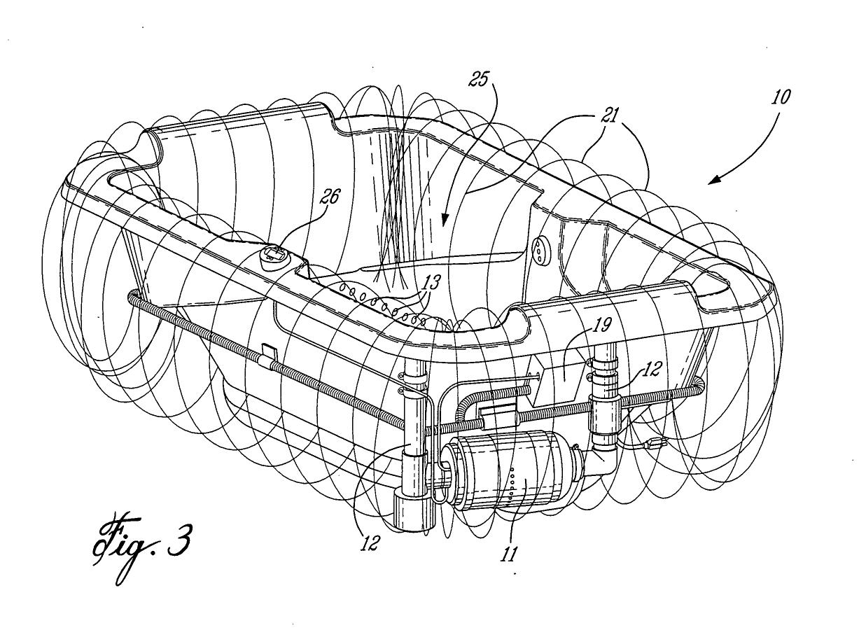

As shown in FIG. 2, the coil 16 is connected to a pulsating voltage generator 20, which is located in the housing 19 also secured to the outer surface 14′ of the circumferential sidewall 14 for generating a pulsating electromagnetic signal into the coil 16. The coil thus generates a pulsating electromagnetic field thereabout, as illustrated by the electromagnetic field pattern 21 in FIG. 3. The generator 20 is controlled by a control circuit 22, which also has a timer device 23 whereby to actuate the generator 20 for a predetermined period of time, usually within the range to 10 to 15 minutes. The generator generates a pulsating voltage at a pulse frequency which does not exceed 60 Hz.

As shown in FIG. 3, the intensity of the pulsating voltage signal flowing through the coil 16 is sufficient to produce an electromagnetic field pattern 21 which can extend only within the treatment space 25′, which is central to the bathtub enclosure. Accordingly, a person sitting in the bathtub 10 will undergo an electromagnetic treatment by the pulsating electromagnetic field and for a predetermined period of time, as set in the control circuit 22. The user person can actuate the system by the remote control 26 accessible to him on the bathtub enclosure at a convenient location.

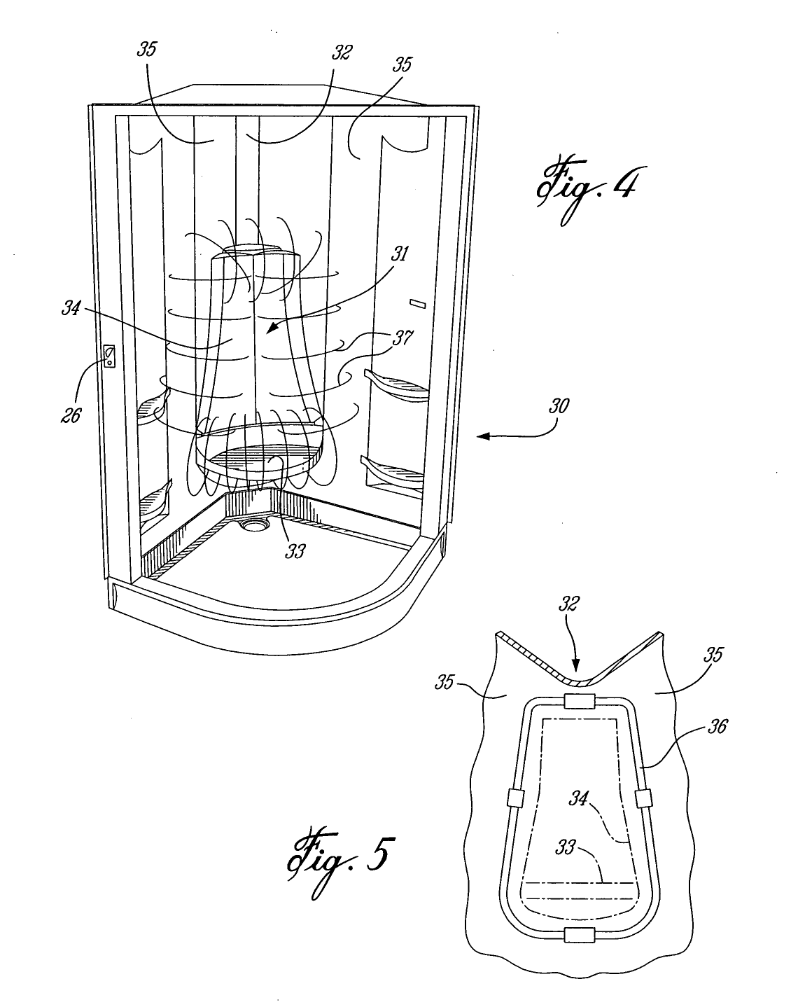

With reference now to FIGS. 4 and 5, it can be seen that a shower enclosure 30 may also be adapted with the electromagnetic treatment system of the present invention. This shower enclosure 30 can be a normal shower enclosure or a therapeutic shower enclosure provided with a therapeutic system not shown herein, but described in a co-pending patent application, and wherein hot dry air is circulated within the enclosure to subject a user person to a therapeutic treatment. As herein shown, the shower enclosure 30 also delineates a treatment space 31 which is herein provided in a corner area 32 of the shower enclosure. The corner area may be fitted with a seat 33 for a person to sit on while undergoing an electromagnetic treatment. Above the sitting area, there is herein shown a backrest portion 34 secured to the side walls 35 of the shower enclosure.

As shown in FIG. 5, the electromagnetic coil 36 is secured in a vertical plane and disposed in alignment from under the seat 33 and about the back rest 34, whereby the electromagnetic field pattern 37 is generated, as shown in FIG. 4, and all about the user person sitting on the seat 33.

Having thus described two examples of the use of the electromagnetic treatment system, the method can be summarized as follows:

A user person positions himself in the treatment area of either the bathtub enclosure 10 or shower enclosure 30 and by the use of the remote control 26, conveniently accessible to him, actuates the voltage generator through the control circuit, whereby the coil generates a pulsating electromagnetic field into the space for the predetermined period of time as dictated by the time device associated with the control circuit. If the bathtub and shower enclosures are therapeutic-type enclosures, then the user person can actuate one or more therapeutic treatment systems prior to or after an electromagnetic treatment system, or in combination therewith (simultaneously), thereby obtaining beneficial effects from these alternative therapeutic treatments. As previously described, the electromagnetic treatment system is beneficial in providing improved blood circulation and the expulsion of lactic acid by the stimulation of the cellular system. It also provides recalcification of bone cells and decalcification of soft tissues. It also neutralizes free radicals and reestablishes the electrical potential of cells. It also stimulates electrochemical cells and releases stress, as well as favoring the absorption of vitamins and minerals, and improves the absorption of molecules such as essential oils. With the therapeutic bathtub enclosure 10, as shown in FIG. 1, if oils are introduced within the water within the bathtub, the stimulation treatment permits the skin to better absorb such oils, thus providing an added benefit to the user person's skin.

It is within the aim of the present invention to provide any obvious modifications of the preferred embodiment described herein, provided such modifications fall within the scope of the appended claims.

We claim:

1. An electromagnetic treatment system in combination with a bathing enclosure adapted to receive a user person, said electromagnetic treatment system comprising an electromagnetic coil attached to an outside surface of a wall of said bathing enclosure and surrounding a space in said enclosure where said user person is disposed to undergo an electromagnetic treatment, a pulsating voltage generator connected to said electromagnetic coil for generating a pulsating electromagnetic field about said coil, said electromagnetic field being oriented relative to said space, control circuit means for controlling said generator, and a remote control secured to said bathing enclosure and accessible to a user person for actuating said generator through said control circuit means.

2. An electromagnetic treatment system as claimed in claim 1, wherein said bathing enclosure is a therapeutic bathing enclosure.

3. An electromagnetic treatment system as claimed in claim 2, wherein said pulsating voltage generator generates a pulsating voltage at a pulse frequency not exceeding 60 Hz.

4. An electromagnetic treatment system as claimed in claim 3, wherein said control circuit means is provided with a timer circuit to control the operational time of said pulsating voltage generator.

5. An electromagnetic treatment system as claimed in claim 4, wherein said operational time is from about 10 to 15 minutes.

6. An electromagnetic treatment system as claimed in claim 2, wherein said therapeutic enclosure is a therapeutic bathtub having massaging jets.

7. An electromagnetic treatment system as claimed in claim 6, wherein said therapeutic bathtub is further provided with one of chromotherapeutic elements on an ozone generator.

8. An electromagnetic treatment system as claimed in claim 6, wherein said electromagnetic coil is a conductive cable secured about a circumferential sidewall of said bathtub in a substantially horizontal plane, said electromagnetic field being oriented transverse to the longitudinal axis of said cable.

9. An electromagnetic treatment system as claimed in claim 3, wherein said therapeutic enclosure is a therapeutic shower enclosure, said enclosure being provided with means delineating said space wherein a user person may position himself to receive an electromagnetic treatment.

10. An electromagnetic treatment system as claimed in claim 9, wherein said treatment space is constituted by a seat in a wall position of said shower enclosure.

11. An electromagnetic treatment system as claimed in claim 10, wherein said coil is a conductive cable secured in a vertical plane and disposed in alignment from under said seat and about a backrest area above said seat.

12. An electromagnetic treatment system as claimed in claim 11, wherein said seat is disposed in a corner area of said shower enclosure, and a molded backrest member secured in said corner area above said seat.

13. A method providing an electromagnetic treatment to a person disposed in a treatment area of one of a bathtub or shower enclosure, said method comprising the steps of:
i) providing such enclosure with an electromagnetic coil attached to an outside surface of a wall thereof and surrounding a space in said enclosure where a user person is disposed to undergo an electromagnetic treatment, and a remote control accessible to said user person;
ii) actuating a pulsating voltage generator by said remote control whereby said coil generates a pulsating electromagnetic field into said space for a predetermined period of time to subject said user person to said electromagnetic treatment.

14. A method as claimed in claim 13, wherein said enclosure is one of a therapeutic bath enclosure, or therapeutic shower enclosure, said method further providing the step of
iii) actuating one or more therapeutic treatments associated with said enclosure prior to or after step (ii).

15. A method as claimed in claim 13, wherein said electromagnetic treatment provides to said user person improved blood circulation and the expulsion of lactic acid through perspiration, stimulates cell regeneration, recalcification of bone cells, decalcification of soft tissues, neutralizes free radicals, reestablish the electrical potential of cells, stimulates electrochemical cells, releases stress, favors the absorption of vitamins, minerals and aligo-elements, and improves the absorption of molecules such as essential oils.
